(19) United States
(12) Patent Application Publication
(10) Pub. No.: US 2009/0090370 A1
(43) Pub. Date: Apr. 9, 2009

(54) PATIENT RESTRAINT SYSTEM

(76) Inventor: Avi J. Bernstein, Wilmette, IL (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 12/246,207

(22) Filed: Oct. 6, 2008

Related U.S. Application Data
(60) Provisional application No. 60/977,807, filed on Oct. 5, 2007.

(51) Int. Cl. A61G 15/00; A61F 5/00; A61B 19/00

(52) U.S. Cl. 128/845; 602/18; 128/869

(57) ABSTRACT

A system for preventing a patient's shoulders from rising or “shrugging” during a medical procedure is presented. The patient lies on a table and wears a jacket. A number of straps are used to couple the jacket to the table. Each strap has a first end and a second end. The first end of each strap is coupled to the jacket and the second end of each strap is coupled to the table. The straps have sufficient tension to prevent the patient's shoulders from rising or “shrugging” during the medical procedure. The patient's feet rest on foot plate to prevent the patient from sliding toward the foot of the table due to the force exerted on the patient's shoulders because of the tension of the straps.

### CROSS-REFERENCE TO RELATED APPLICATION

This application claims the benefit of U.S. Provisional Patent Application No. 60/977,807, filed Oct. 5, 2007, the entire contents of which are incorporated by reference herein.

### BACKGROUND

To provide medical personnel and equipment with appropriate access to a patient, the position of the patient's body is often manipulated either before or during a medical procedure. For example, during surgical procedures involving the cervical thoracic junction of the patient's spine (i.e., the neck), medical personnel often use x-rays and fluoroscopy to obtain images of the internal structure of the patient's neck. To obtain a clear image, it is important that the medical personnel and equipment have an unrestricted view of the patient's neck. However, often during such procedures, the patient's shoulders rise toward the patient's head or “shrug,” causing the shoulders to partially cover the patient's neck and interfere with the x-ray or fluoroscopy machines, as well as other activities of the medical personnel.

One solution to this problem is to have additional medical personnel hold the patient's shoulders in place during the surgical procedure. However, these additional medical personnel add further expense and occupy precious space around the patient, thereby contributing to the problem of obtaining an unobstructed view of the neck. Another solution is to tape the patient's shoulders to the operating table or use two empty water bottles to press the patient's shoulders so that they cannot rise toward the patient's head. However, the tape can become detached from the patient's shoulders or the table, and the water bottles may shift during the operation, allowing the patient's shoulder's to rise.

### SUMMARY

Accordingly, there is a desire to have a device that is capable of restraining a patient's shoulders without the need for additional medical personnel, tape, or the use of empty water bottles during a surgical procedure.

To this end, embodiments of the present invention disclose a garment, such as a jacket or vest that a patient wears during a medical procedure, such as surgery. The jacket may be placed on the patient prior to or during the medical procedure. The jacket has an anterior portion and a posterior portion which are attached above the patient's shoulders.

During the medical procedure, the patient lies on an operating table that has a head portion (i.e., the end of the table that is closest to the patient's head) and a foot portion (i.e., the part of the table that is closest to the patient's feet). The jacket is coupled to the table with a number of straps (i.e., four). The first end of each strap is coupled to the jacket and the second end of each strap is coupled to the operating table at a location that is closer to the foot portion of the table than the head portion.

The tension of the straps may be adjusted so that they exert a force on the jacket in the general direction of the foot portion of the operating table. This force prevents the patient's shoulders, which are restrained inside of the jacket, from rising or “shrugging.” In addition, the patient's feet are secured to a foot plate which projects vertically up from the operating table, allowing the patient to resist the force applied by the straps to the jacket.

### BRIEF DESCRIPTION OF THE DRAWINGS

FIG. 1 is a front perspective view of the patient restraint system in accordance with one embodiment;

FIG. 2 is a top view of the patient restraint system in accordance with a second embodiment;

FIG. 3 is a top view of patient restraint system in accordance with a third embodiment.

FIG. 4 is a side view of a foot rest that is used in connection with the patient restraint system of FIG. 1.

FIG. 5 is a front perspective view of the patient restraint system in a fourth embodiment.

FIG. 6 is a front perspective view of the left anterior strap of the patient restraint system of FIG. 5.

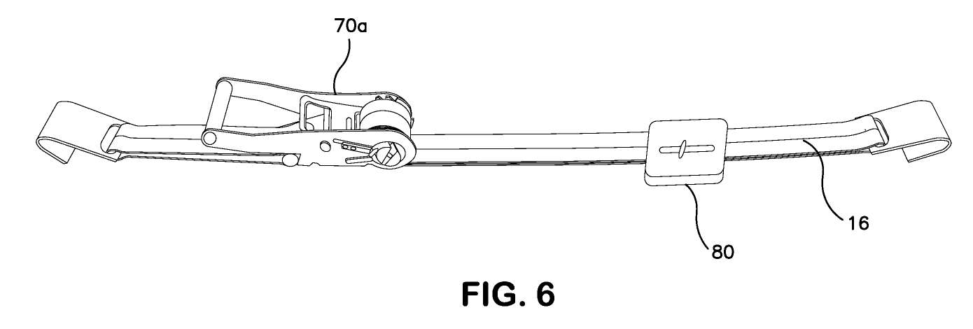

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

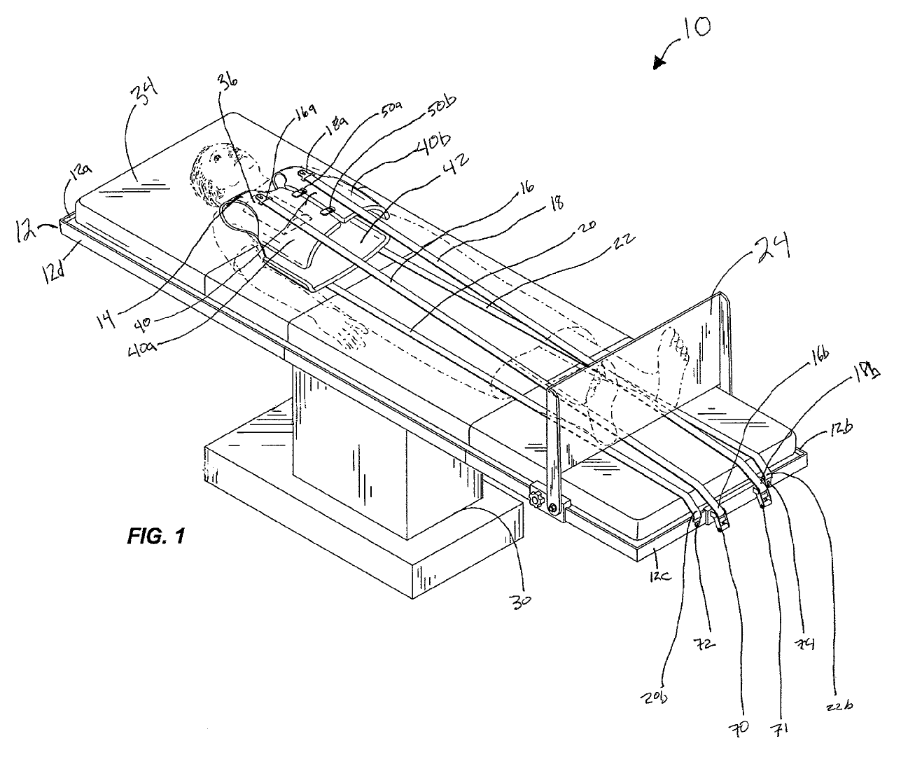

FIG. 1 depicts a system 10 that is configured to maintain a patient's neck in a position so as to provide an unobstructed view during a medical procedure, such as surgery. In the illustrated embodiment, the system 10 includes a support table 12, a garment such as a jacket 14, a number of straps (e.g., four as shown) 16, 18, 20, 22, and a foot board 24. The support table 12 supports the patient's body during a medical procedure. The support table 12 is a generally flat structure of the type that is usually used to support a patient's body, as well as any medical equipment that may be coupled to or placed upon the support table 12, during a medical procedure. The support table 12 is supported and elevated from the floor by a base 30. In addition, the support table may include a mattress 34 to provide further support to the patient's body. In the illustrated embodiment, the support table 12 is generally rectangular in shape having a head 12a, a left side 12b, a foot 12c, and a right side 12d.

In addition, a semi-cylindrical roll 36 is placed on the mattress 34. The semi-cylindrical roll is placed with the flat side down while the arch is placed longitudinally beneath the patient's upper thoracic spine (e.g., shoulder blades) when the patient is lying in a supine position, allowing the patient's shoulder blades to retract. The roll 36 may be constructed of foam or any other material suitable for providing support to the patient's spine.

As depicted in FIGS. 1-3, the system 10 also includes a jacket 14 which the patient wears during the medical procedure. The jacket 14 is placed upon the patient either before or during the medical procedure. The jacket 14 is coupled to the support table 12 via a set of straps 16, 18, 20, 22 as described below. The jacket 14 includes an anterior portion 40 and a posterior portion 42. The jacket 14 may be constructed of foam, vinyl, cloth, leather, or any other material or combinations of material of sufficient strength to withstand the force that is exerted on the jacket 14 by the straps 16, 18, 20, 22 as described below. The anterior portion 40 of the jacket 14 fits over the patient's chest. In the illustrated embodiment, the anterior portion 40 is separated into a right anterior portion 40a and a left anterior portion 40b (“right” and “left” are designated from the perspective of the patient). In other embodiments, the anterior portion 40 of the jacket 14 is formed as one solid portion that fits over the patient's chest. In the illustrated embodiment, the right anterior portion 40a and the left anterior portion 40b are coupled together across the patient's chest with a number (e.g., two as shown) of clips 50a, 50b. In other embodiments, the right anterior portion 40a and the left anterior portion 40b may be coupled together using buckles, snaps, ties, hook and loop fasteners, or any other means which are suitable for coupling two portions of a jacket together. In still other embodiments, the right anterior portion 40a and the left anterior portion 40b are not coupled together and lie side by side while the jacket 14 is in use.

The posterior portion 42 of the jacket 14 fits over the patient's back and lies between the mattress 34 and the patient's back when the patient is lying in a supine position.

The anterior portion 40 and the posterior portion 42 of the jacket 14 are coupled together over the patient's shoulders. The anterior portion 40 and the posterior portion 42 may be coupled together by stitching, snaps, clips, hook and loop fastener or any other means suitable for connecting the front and back of a jacket. The patient's head fits through a hole 45 in the top of the jacket 14.

In the illustrated embodiment, the jacket 14 does not completely cover the sides of the patient's torso, thereby making the jacket 14 very easy to apply to a supine patient. In other embodiments, the anterior portion 40 and posterior portion 42 of the jacket 14 are coupled together on the sides of the patient's torso so that the jacket 14 completely covers the patient's torso. In this embodiment, the patient's arms fit through holes in the side of the jacket 14.

In the illustrated embodiment, the jacket 14 is coupled to the support table 12 with two anterior straps 16, 18 and two posterior straps 20, 22. The straps 16, 18, 20, 22 may be constructed from nylon, plastic, leather, or any other material of sufficient tensile strength to restrain the patient's shoulders as described below. Each of these straps has a first end 16a, 18a, 20a, 22a and a second end 16b, 18b, 20b, 22b. The first ends of the anterior straps 16a, 18a are coupled to the anterior portion 40 of the jacket 14. The first anterior strap 16 is coupled to the right anterior portion of the jacket 40a and the second anterior strap 18 is coupled to the left anterior portion 40b of the jacket. The first ends of the anterior straps 16a, 18a may be coupled to the jacket 14 using hooks, buckles, buttons, snaps, hook and loop fasteners, stitching, or any other method suitable for securing straps to a jacket.

The first ends of the posterior straps 20a, 22a are coupled to the posterior portion 42 of the jacket 14. The first posterior strap 20 is coupled to the right side of the posterior portion 42 and the second strap 22 is coupled to the left side of the posterior portion 42 of the jacket 14. The first ends of the posterior straps 20a, 22a are coupled to the jacket 14 using the same method described above with respect to the first ends of the anterior straps 16a, 18a.

The second ends of the anterior straps 16b, 18b and the posterior straps 20b, 22b are coupled to the support table 12. In the illustrated embodiment, the anterior straps 16b, 18b are coupled to the foot of the support table 12c with a pair of tension clamps 70, 71. The tension clamps 70, 71 may be temporarily coupled (e.g., clamped) or permanently coupled to the support table 12. The tension clamps 70, 71 have an open and a closed position. As shown in FIG. 1, the anterior straps 16, 18 are configured such that when the clamps 70, 71 are in the open position, there is relatively little tension on the anterior straps 16, 18 so that the jacket 14, position of the patient, or position of the foot board 24 may be adjusted. However, when the clamps 70, 71 are in their closed position (not shown), there is enough tension on the anterior straps 16, 18 to exert a force on the jacket 14, and the patient's shoulders which are restrained inside of the jacket 14, in the direction of the foot of the support table 12c.

The second ends of the posterior straps 20b, 22b are coupled to foot of the support table 12c. As depicted in FIG. 1, the second ends of the posterior straps 20b, 22b are coupled to the support table with a pair of hooks 72, 74. There is enough tension on the posterior straps 20, 22 to exert a force on the jacket 14, and the patient's shoulders which are restrained inside of the jacket 14, in the direction of the foot of the table 12c. The combined force of the anterior straps 16, 18 (when the tension clamps 70, 71 are in the closed position) and the posterior straps 20, 22 prevent the patient's shoulders from rising or “shrugging” during the medical procedure.

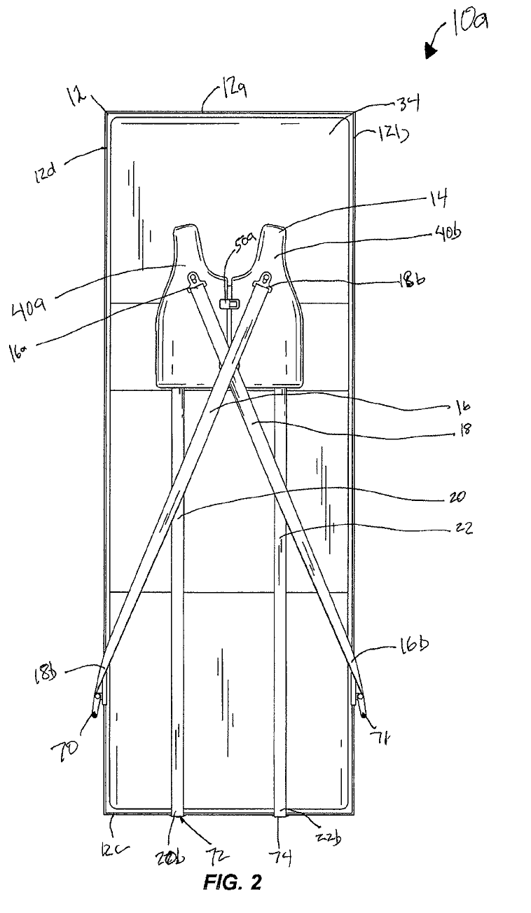

FIG. 2 is a depiction of an alternative embodiment wherein the system 10a includes anterior straps 16, 18 that cross in front of the patient's body during the medical procedure. In this alternative embodiment, the first anterior strap 16, which is coupled to the right anterior portion 40a of the jacket 14, is coupled to the clamp 70 located on the left side of the support table 12b and the second anterior strap 18, which is coupled to the clamp 71 left anterior portion 40b of the jacket 14, is coupled to the right side of the support table 12d. In this embodiment, the second ends of the anterior straps 16b, 18b are coupled to the sides of the support table 12b, 12d, at a location that is closer to the foot of the support table 12c than the head of the support table 12a, using tension clamps 70, 71 in the same manner described above with regard to the embodiment of FIG. 1. The posterior straps 20, 22 are coupled to the jacket 14 and to the support table 12 in the same manner as described above with regard to the embodiment of FIG. 1.

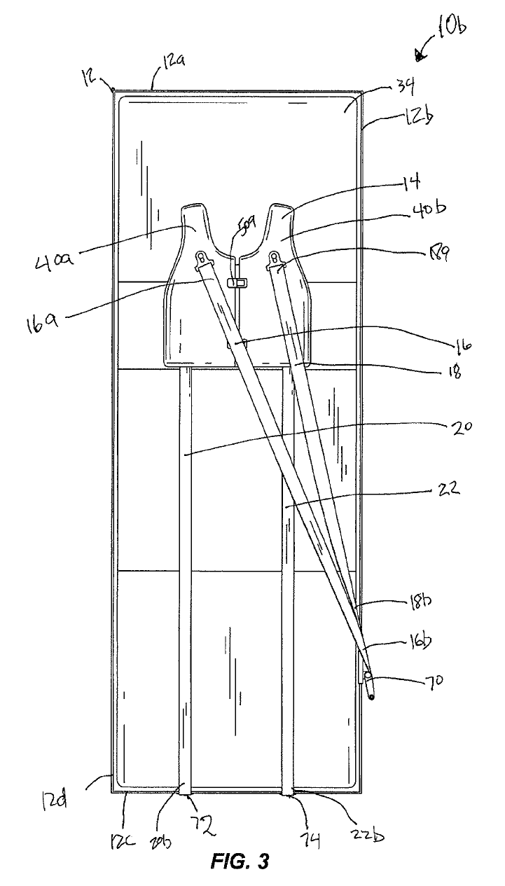

FIG. 3 is a depiction of another alternative embodiment wherein the system 10b includes anterior straps 16, 18 that are coupled to only one side of the support table 12 (e.g., the left side 12b as shown). In this alternative embodiment, the first anterior strap 16, which is coupled to the right anterior portion 40a of the jacket 14, and the second anterior strap 18, which is coupled to the left anterior portion 40b of the jacket 14, are coupled to the left side of the support table 12b. The second ends of the anterior straps 16b, 18b are coupled to the left side of the support table 12b, at a position that is closer to the foot of the support table 12c than the head of the support table 12a, using a tension clamp 70 in the same manner described above with regard to the embodiment of FIG. 1. The posterior straps 20, 22 are coupled to the jacket 14 and to the support table 12 in the same manner as described above with regard to the embodiment of FIG. 1. Other embodiments of the invention may include different numbers or configurations of straps, provided that the number of straps and their configuration provide sufficient force on the cervical junction jacket 14 to restrain the patient's shoulders.

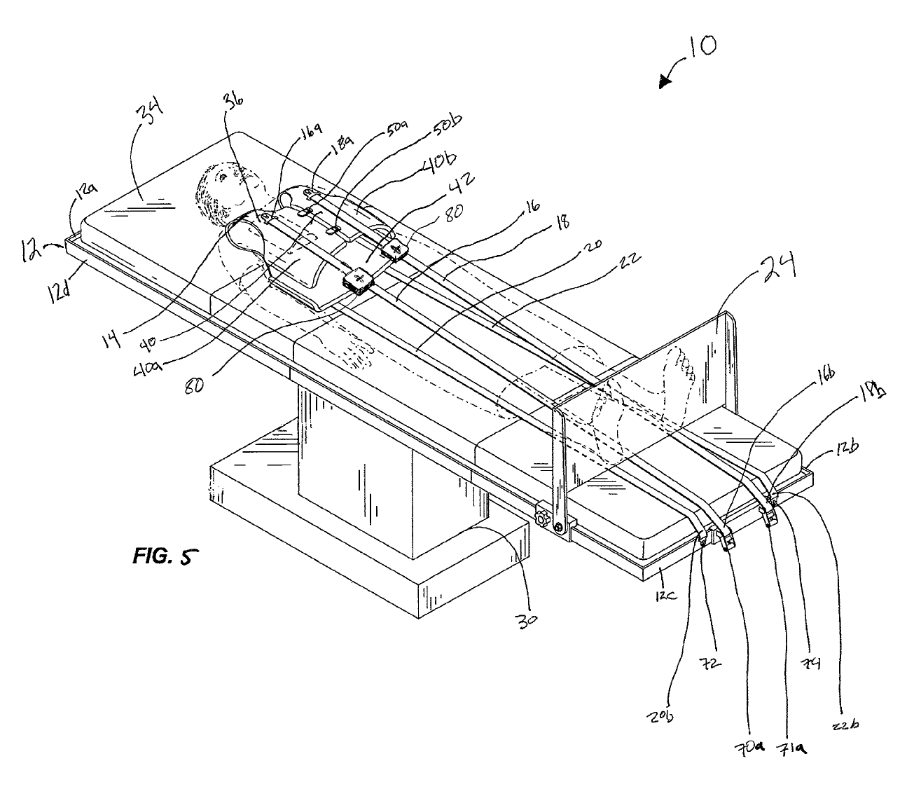

FIG. 5 is a depiction of an additional embodiment, wherein the second ends of the anterior straps 16b, 18b and posterior straps 20b, 22b are coupled to an anchor point using one or more winches 70a, 71a. The winch may include a roller, a crank arm to turn the roller, and a release. In this embodiment, the winch is securely coupled to the support table 12 either permanently or by some temporary means (e.g., a clamp). Further, the crank arm may be used to advance the roller in only-a single direction, and to reverse the roller a release must be set. As depicted in FIG. 5, the winch may be coupled to the foot of the support table 12c. The winch accepts at least one of the second ends of the straps 16b, 18b, 20b, 22b such that the second end of the strap is at least partially wound around the roller. The crank arm advances the roller to further wind the strap until there is sufficient tension to prevent the patient's shoulders from rising or “shrugging” during the medical procedure. The release is used when the medical procedure is complete, allowing the rollers to rotate in a direction which unwinds the straps, causing them to loosen.

As also depicted in FIG. 5, at least one of the straps 16, 18, 20, 22 is configured with a tension meter 80. The tension meter is connected to at least one of the straps 16, 18, 20, 22 and measures the tension thereon. The tension meter thus allows the medical personnel to ensure the strap maintains sufficient tension to prohibit the patient's shoulders from rising or “shrugging,” while simultaneously ensuring the tension on the straps 16, 18, 20, 22 is not excessive as to injure the patient.

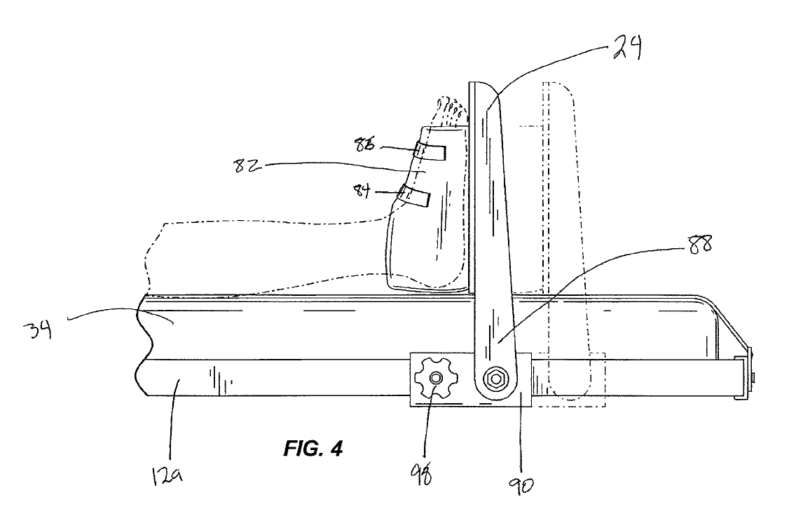

FIGS. 1 and 4 depict the foot plate 24. The foot plate 24 is adjustably coupled to the support table 12. The foot plate 24 projects vertically upward from the support table 12, supporting the patient's feet and providing resistance to the force applied on the patient's body by the straps 16, 18, 20, 22. In the embodiment of FIG. 4, the foot plate 24 includes two boots 82 which are fitted to the patient's feet and secure the patient's feet to the foot plate 24. The boots 82 may be constructed of foam, cloth, leather, plastic, or any other material of sufficient strength to support the patient's foot. Further, as depicted in FIG. 4, the boots 82 may include a number (e.g., two as shown) of fasteners 84, 86 which further secure the patient's foot inside of the boot 82. These fasteners 84, 86 may be constructed of hook and loop fasteners, leather, plastic, metal, or any other material or combination of materials suitable for the construction of a fastener. In other embodiments, the boots 82 may not include any fasteners. As depicted in FIG. 1, the patient's feet may also rest directly on the foot plate 24. Where the patient's feet rest directly on the foot plate 24, a textured surface may be used to allow the patient's feet to grip the foot plate 24.

As depicted in FIG. 1, the foot plate 24 extends from the left side of the support table 12b to the right side of the support table 12d. Each side of the foot plate 40 includes a vertically extending arm 88 which is coupled to a foot plate mount 90. FIGS. 1 and 4 depict only a vertically extending arm 88 and foot plate mount 90, both on the right side of the foot plate 24, but the left side of the foot plate 24 is also configured with a vertically extending arm and a foot plate mount. The right foot plate mount 90 is coupled to the right side of the support table 12d and the left foot plate mount (not shown) is coupled to the left side of the support table 12b. As depicted in FIG. 4, the right foot plate mounts 90 slides laterally along the right side 12d of the support table, and because the right foot plate mount 90 and the left foot plate mount (not shown) are both coupled to the foot plate 24, all three components slide laterally as one unit on the support table 12. This sliding movement allows the foot plate 24 to be adjusted to the height of the patient.

As depicted in FIG. 4, the right side mount 90 includes a locking knob 98. The locking knob 98 may be rotated in either direction. When the locking knob 98 is rotated in a first direction, the right side mount 90, left side mount, and the foot plate 24, are locked in a fixed position with respect to the head of the support table 12a. When the locking knob 98 is rotated in the opposite direction, the right side mount 90, left side mount, and the foot plate 24, are released so that their position with respect to the head of the support table 12a may be adjusted. In some embodiments, the left side mount may also include a locking knob which functions in the same manner. If both the right side mount 90 and the left side mount include a locking knob 98, both locking knobs must be released in order to adjust the position of the foot plate 24. In other embodiments, the right side mount 90 or left side mount may be secured to the sides of the support table 12 using a clamp, pins, or any other method suitable for locking the foot plate 24 to the support table 12.

1. A patient restraint for maintaining a patient's neck position during a medical procedure that occurs while the patient is supported on a table, the patient restraint comprising:
a garment placeable on the patient's body; and
at least one strap, each strap having a first end coupled to the restraint and a second end capable of coupling to an anchor point.

2. The patient restraint of claim 1, and the anchor point substantially located on the table.

3. The patient restraint of claim 1, wherein the garment further comprises:
an anterior portion for locating near the chest of the patient;
a posterior portion connected to the anterior portion and for locating near the back of the patient; and
a recess between the anterior and posterior portions and configured to allow clearance for the patient's head.

4. The patient restraint of claim 3, wherein the anterior portion includes a left anterior portion and a right anterior portion, and wherein the left and right anterior portions can be coupled together.

5. The patient restraint of claim 4, wherein the garment further comprises a plurality of clips for coupling the left and right anterior portions.

6. The patient restraint of claim 3, wherein the anterior portion and the posterior portion are connected by a pair of shoulder straps.

7. The patient restraint of claim 1, further comprising a foot plate couplable to the table.

8. The patient restraint of claim 7, wherein the foot plate comprises:
a left foot plate mount and a right foot plate mount, the left and right foot plate mounts slidably couplable to the support table; and
a pair of arms extending from the foot plate and coupled to the foot plate mounts.

9. The patient restraint of claim 8, wherein at least one of the left foot board mount and right foot plate mount further comprise a locking knob, wherein the locking knob is engageable to prohibit translative motion of the foot board mount.

10. The patient restraint of claim 7, wherein the foot plate further comprises a plurality of boots for securing the patient's feet to the foot plate.

11. The patient restraint of claim 1, further comprising a force meter coupled to the at least one strap.

12. The patient restraint of claim 1, further comprising a tensioning device, and wherein the tensioning device is positioned to apply an adjustable force along the at least one strap.

13. The patient restraint of claim 12, wherein the tensioning device is a winch.

14. A method for restraining the shoulders of a patient, the method comprising:
placing the patient on a support table;
placing a garment on the patient;
coupling a first end of the at least one strap to the restraint;
coupling a second end of the at least one strap to an anchor point;
adjusting a foot plate to contact the patient's feet; and
applying tension to the at least one strap.

15. The method of claim 14, wherein coupling the second end of the strap to an anchor point further comprises anchoring the second end of the strap to the support table.

16. The method of claim 14, wherein placing the patient on a support table further comprises placing the patient in a supine position on the support table.

17. The method of claim 14, wherein placing the restraint on the patient further comprises placing a garment on the patient, the garment comprising a left anterior portion, a right anterior portion, and a posterior portion.

18. The method of claim 17, further comprising:
placing the posterior portion near the back of the patient;
placing the left and right anterior portions on the chest of the patient; and
coupling the left anterior portion and the right anterior portion.

19. The method of claim 14, the method further comprising placing a semi-cylindrical roll substantially near the patient's spine to facilitate retracting the patient's shoulder blades.

20. The method of claim 14, wherein applying tension to the at least one strap further comprises utilizing a winch.

21. The method of claim 20 and measuring the tension in the at least one strap.
